(19) United States
(12) Patent Application Publication
(10) Pub. No.: US 2004/0267219 A1
(43) Pub. Date: Dec. 30, 2004

(54) Semi-disposable and/or non-disposable diaper system for babies and adults

(76) Inventor: Magdalena Luisa Olmedo, Buenos Aires (AR)

Correspondence Address: CLARK & BRODY, Suite 600, 1750 K Street, N.W., Washington, DC, 20006, US

(21) Appl. No.: 10/697,885

(22) Filed: Oct. 31, 2003

(30) Foreign Application Priority Data
Nov. 14, 2002 (AR) P020104388

(51) Int. Cl. A61F 13/15; A61F 13/20

(52) U.S. Cl. 604/367; 604/385.24

(57) ABSTRACT

A semi-disposable and/or non disposable diaper system for babies and adults comprises a non-disposable diaper cover, made of PVC and polypropylene completed by a double diaper system. The double diaper system comprises one non-disposable diaper made of layers of gauze one on top of the other or of layers of gauze and absorbent material of polypropylene. Another disposable diaper of the double diaper system is made of cellulose and super-absorbent polymer. Both diapers are designed in an anatomical shape and size that together with the diaper cover work as only one piece.

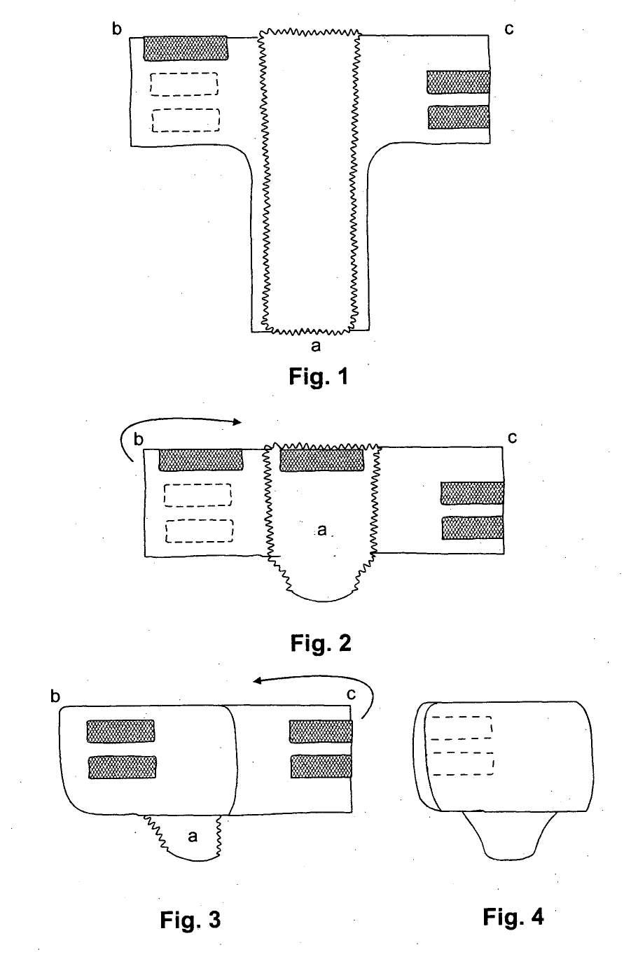

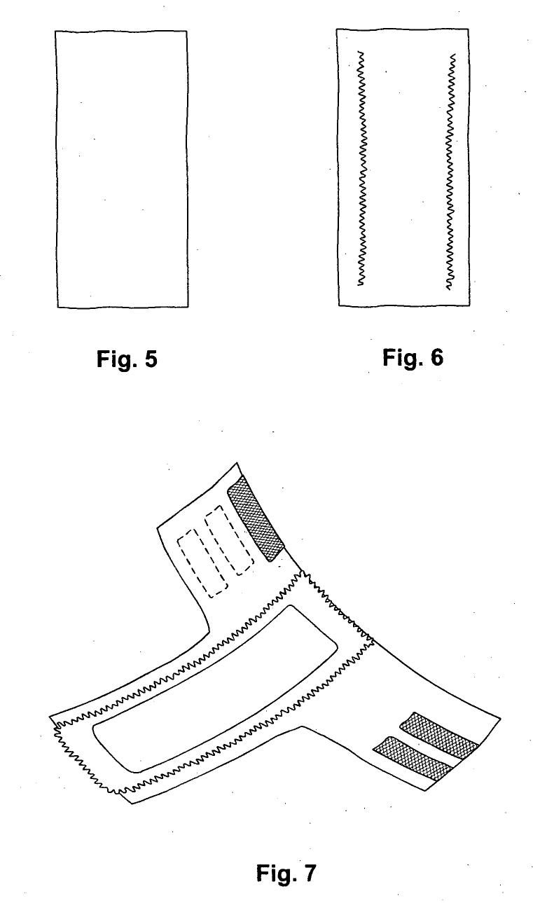

### TECHNICAL FIELD OF THE INVENTION

This invention refers to a semi-disposable or non-disposable diaper system for babies and adults, by means of which it is possible not only to simplify the use but also to lower the cost as it allows to use different kinds of non-perishable materials (non-disposable option) or disposable materials of long durability (semi-disposable option) in a conjunction that assures the protection of either baby or adult's hygiene in a practical and efficient way.

### STATE OF THE TECHNIQUE AND PROBLEMS TO SOLVE

The complexity of the traditional diaper system (square, made of gauze, covered by an extra T shape diaper made of flannel adjusted to the baby's waist by means of strips and protected by rubber panties) imposed the system of completely disposable diapers with the high cost that this system means in the family budget.

At present, it is known a large variety of this completely disposable diaper system which was thought to make the use practical and effective.

By means of this invention the same benefits are obtained at a much lower cost, as it gives the possibility of choosing between the non-disposable or the semi-disposable systems.

### OBJECT OF THE INVENTION

The aim sought by the invention is obtained by means of two alternatives: A semi-disposable one that provides a quick and practical use at a lower cost, as it is not completely disposable. A non-disposable one: with which the maximum economic benefit is obtained, as it allows in an easy and quick way the whole recovery after each use.

### BRIEF DESCRIPTION OF THE DRAWINGS

Picture 1: Open view of the diaper cover, inner side with the adjustable Velcro grip tabs and elastic leggings.

Picture 2: View of A overlapping.

Picture 3: B overlapped in A according to the Velcro line that appears in Picture 2.

Picture 4: Diaper cover closed.

Picture 5: Disposable or non-disposable rectangular flannel with straight borders.

Picture 6: Disposable or non-disposable rectangular flannel with elastic borders with or without leak guard system.

Picture 7: Open view of how to place the diaper (disposable or not) over the diaper cover.

### DETAILED DESCRIPTION OF THE INVENTION

This invention includes: A—A diaper cover made of PVC. Non-disposable, comfortable and washable, adjustable by means of Velcro grip tab, and elastic system to the baby's body.

There exist two possible alternatives:

Alternative A-1: It is made of a double base of PVC (solid or print) with a T-shape and between both layers of PVC it is placed another layer made of an un-woven fabric of polypropylene. This is made by means of two rectangles that cross each other, as it was said, forming a “T”. The upper rectangle is adjusted to the baby's waist and it is placed above the lower rectangle that covers the crotch from back to front. Around the baby's legs some elastic is placed with or without leak guard system, that holds and fastens the diaper in a panty-like fashion to fit the baby's body perfectly. The diaper is adjusted by means of adjustable Velcro grip tabs to ensure a perfect fit for every size baby, offering comfort and freedom of movement.

Alternative A-2: Following the same procedure, the difference appears in the combination of materials; as it is made of a double PVC base only on the lateral ends. In the middle an absorbent fabric delimited by elastic, with or without leak guard system is placed over the PVC base.

The diaper cover is completed by:

B—A practical and absorbent rectangular diaper made of several layers of gauze or of several layers of gauze and super-absorbent fibre of polypropylene sheets, which is easy to wash and dries fast. These last qualities allow to achieve the sought aim, a practical and effective use, and in this stage of the inventive system it is also obtained the highest economic benefit in the use of diapers as it allows the recovery of not only the diaper cover but also of the absorbent diaper made of gauze and polypropylene, which are both easy to wash and dry fast.

It is made of layers of gauze, that enclose inside, a super-absorbent fibre, in an anatomical shape that is adapted to the diaper cover in its middle and lower part.

Two alternatives are also possible here: In the first one the rectangle is delimited in a simple way, in the other the borders are delimited by elastic and absorbent material, forming a leak guard system.

C—This set is completed by a disposable flannel (semi-disposable alternative) made of a cellulose material and absorbent gel, covered by polypropylene, to prevent the baby from getting scalded. This diaper due to its simple characteristics is extremely practical and much more inexpensive than the completely disposable ones. As in option B, it is made in a rectangular and anatomical shape that is adapted to the diaper cover in its middle and lower part, with leak guard system in the lateral borders. Here it is also possible the second alternative of delimiting the borders in a straight way or with elastic as a second option.

The combination of both alternatives, B or C, in the family use, fits the aim of the inventive that is to provide, with a practical and effective use, an economic benefit according to the individual needs, thanks to the free election of options that the system offers.

Replevins

Having described and specified the nature and scope of the invention and the way to put it into practice, it is hereby replevied the exclusive right and property of:

1. The semi-disposable and/or non disposable diaper system for babies and adults, which includes a non-disposable diaper cover, made of PVC and polypropylene as it is described in item A of the “Detailed Description of the Invention” completed by a double diaper system: one that is non-disposable, made of layers of gauze one on top of the other or of layers of gauze and absorbent material of polypropylene and another made of disposable material of cellulose and super-absorbent polymer. Both are designed in an anatomical shape and size that together with the diaper cover work as only one piece and make its use practical (as it is described in items B and C of the “Detailed Description of the Invention”).

2. To replevy, independently, the panty diaper or diaper cover system, in the materials stated or in those that could be adapted to improve the system, according to the needs that in practice the use determines.

3. To replevy, independently, its complementary diaper, made of gauze and absorbent material, with or without a leak guard system, straight or with elastic leggings and all the improvements that could be made to obtain a better quality in the use, as long as they adjust to the invention that is registered.

4. To replevy, independently, the system of disposable flannels, with or without leak guard system, straight or with elastic leggings and its treatment with protective gels or creams, to affect the use of the semi-disposable system that is registered.

1-4. canceled

5. A semi-disposable and/or non disposable diaper system for babies and adults comprising:
a non-disposable diaper cover, made of PVC and polypropylene completed by a double diaper system, the double diaper system further comprising:
one non-disposable diaper made of layers of gauze one on top of the other or of layers of gauze and absorbent material of polypropylene, and
one disposable diaper made of cellulose and super-absorbent polymer,
wherein both diapers are designed in an anatomical shape and size that together with the diaper cover work as only one piece.

6. The diaper system of claim 5, wherein the non-disposable diaper made of gauze or gauze and absorbent material includes one or more of the following:
straight sides without elastic leggings; or
elastic leggings.

7. The diaper system of claim 5, wherein the disposable diaper comprises a disposable flannel, with one of the following:
straight sides without elastic leggings, or
elastic leggings.

8. The diaper system of claim 7, wherein the disposable flannel is treated with protective gels or creams.

9. The diaper system of claim 6, wherein the non-disposable diaper includes a leak guard system.

10. The diaper system of claim 7, wherein the disposable diaper include a leak guard system.
